(19) United States
(12) Patent Application Publication
(10) Pub. No.: US 2005/0177073 A1
(43) Pub. Date: Aug. 11, 2005

(54) Guidewire system with a deflectable distal tip

(76) Inventor: Samuel Shiber, Manchester, NH (US)

Correspondence Address: SAMUEL SHIBER, 365 KEARNEY CR, MANCHESTER, NH, 03104, US

(21) Appl. No.: 11/097,921

(22) Filed: Apr. 1, 2005

Related U.S. Application Data
(63) Continuation-in-part of application No. 11/071,140, filed on Mar. 3, 2005.
(63) Continuation-in-part of application No. 10/937,134, filed on Sep. 9, 2004.
(63) Continuation-in-part of application No. 10/620,740, filed on Jul. 16, 2003.
(63) Continuation-in-part of application No. 10/463,189, filed on Jun. 17, 2003.

(51) Int. Cl. A61B 5/00; A61M 25/00

(52) U.S. Cl. 600/585

(57) ABSTRACT

A flexible guidewire system with a deflectable distal tip comprising a flexible tubular shield adapted to slide and rotate over a pilot wire, a flexible casing disposed over the shield, at least a distal portion of the casing being a helical wire, a first coupling means connected to the casing, a second coupling means connected to the shield and a thin narrow flexible link disposed in the distal portion of the casing, adjacent to one side of an inner surface of the casing, that connected to the distal end of the casing and to a distal end of the shield and compresses, to a larger extent on the one side that is adjacent to the link, when the first coupling means is displaced relative the second coupling means causing the distal portion of the casing to curve and deflect the distal tip.

### CROSS REFERENCE TO RELATED APPLICATIONS

This application is a continuation-in-part of my co-pending applications Ser. No. 11/071140 that was filed on Mar. 3, 2005 (CT25), Ser. No. 10/937134 that was filed on Sep. 9, 2004 (CT24), Ser. No. 10/620740 that was filed on Jul. 16, 2003 (CT23) and Ser. No. 10/463189 that was filed on Jun. 17, 2003 (CT22).

All of the above are being incorporated herein by reference.

### BACKGROUND AND OBJECTIVES OF THE INVENTION

With age a large percentage of the population develops atherosclerotic and thrombotic obstructions resulting in partial or total occlusions of blood vessels in various parts of the human anatomy. Such obstructions are often treated with angioplasty or atherectomy catheters and a common preparatory step to such procedures is the insertion of a guidewire through the obstruction.

An objective of the present invention is to provide a simple and reliable flexible guidewire system with a deflectable distal tip capable of navigating through tortuous vasculature and of crossing obstructions, particularly tight and total obstructions.

The above and other objectives of the invention will become apparent from the following discussion and the accompanying drawings.

### BRIEF DESCRIPTION OF THE FIGURES

FIG. 1 schematically shows a side view of a flexible guidewire system with a deflectable distal tip, for crossing an obstruction in a vessel and/or for navigating through tortuous vasculature, being inserted over a guidewire from a patients groin area through his arterial system into his obstructed coronary artery;

FIG. 2 shows a proximal portion of the system;

FIGS. 3 and 3′ show a side and front views, respectively, of a distal portion of the system;

FIG. 4 shows a side view of the system disposed over a guidewire;

FIG. 5 shows a deflectable distal tip;

FIGS. 6 and 6′ show a side and distal end views, respectively, of the system disposed in a flexible sleeve having an inflatable distal chamber; and

FIGS. 7 and 7′ show the deflectable distal tip of an embodiment in which the casing is wound of a flattened wire.

### DETAILED DESCRIPTION OF THE DRAWINGS

The FIGURES show a flexible guidewire system with a deflectable distal tip made of elongated components that are rotatable and slidable one relative to the other (the components' ends that go further into the vessel are referred to as “distal” and their other ends are referred to as “proximal”). The system is shown disposed over a pilot wire 9 across an obstruction 12 located in a patient's coronary vessel 13 serving the heart 11 (the patient's anatomy and the system are illustrated schematically and are not drawn to scale).

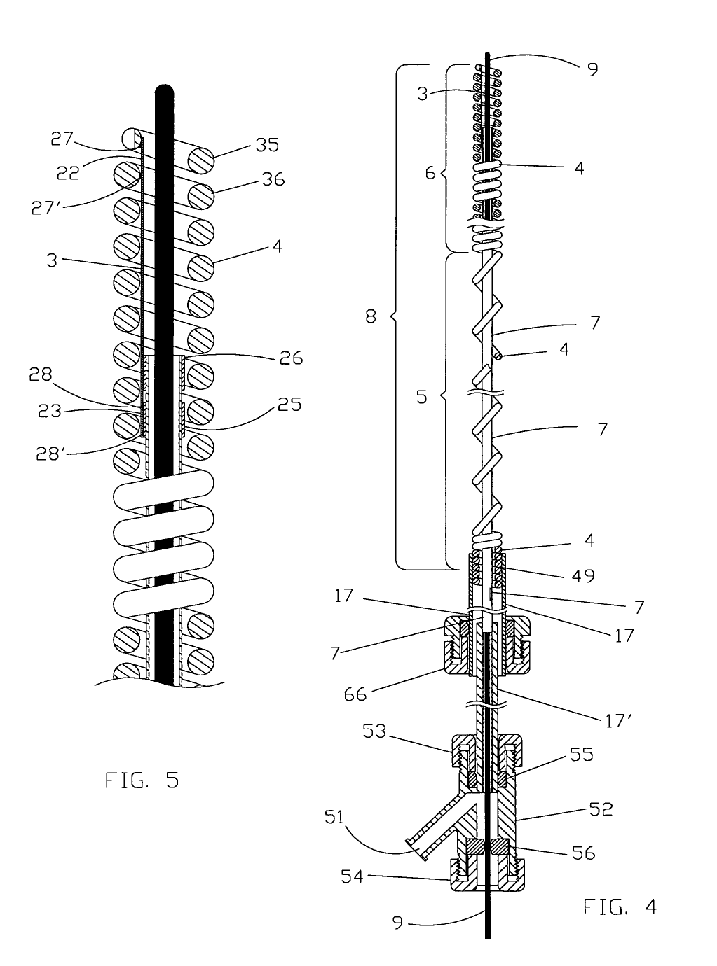

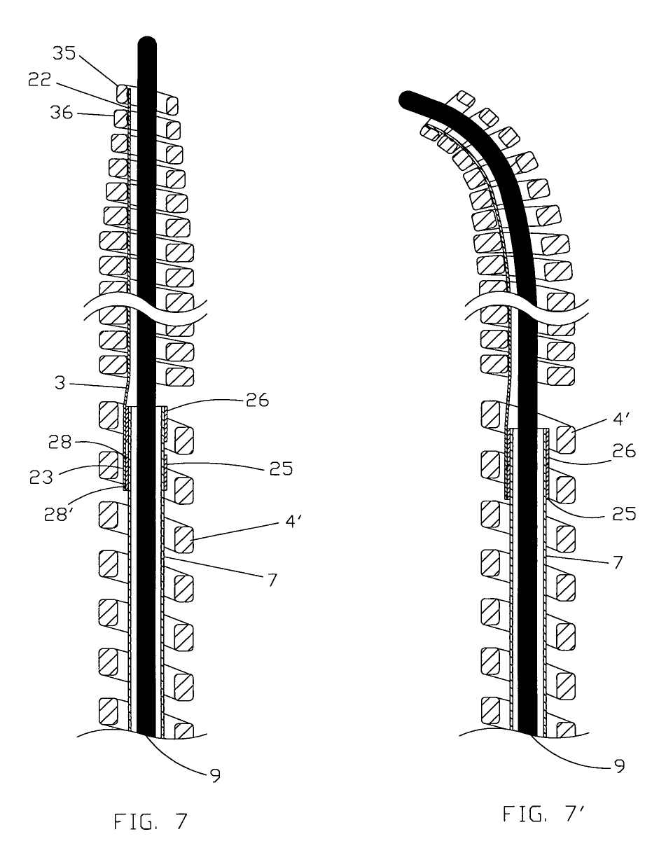

The system 10 comprises a flexible tubular shield 7 (note FIG. 4) adapted to slide and rotate over the pilot wire 9. The flexible pilot wire can be a standard guidewire (guidewires are sold by numerous companies, e.g.: Boston Scientific, Natick, Mass.; Cook, Bloomington, Ind.). A flexible casing 8 is disposed over the shield. At least a distal portion of the casing 6 being a helical wire. A first coupling means 17 is connected to the casing, preferably by a weldment 49. A second coupling means 17′ is connected, preferably by an adhesive, to the shield. A thin narrow flexible link 3 is disposed in the distal portion of the casing 6 adjacent to one side of an inner surface of the distal portion of the casing. The link has a distal and proximal ends 22 and 23 that are connected to a distal end of the casing and to a distal end of the shield, respectively. The link is designed to pull and compresses the distal portion of the casing 6 in response to the first coupling 17 means being displaced relative the second coupling means 17′, which in turn displaces the shield 7 relative to the casing 8, causing the helical wire to compress to a larger extent on the one side that is adjacent to the link thereby causing the distal portion of the casing to curve and deflect the distal tip of the casing, as shown in FIG. 7′. The ability to selectively deflect the tip enables the user to navigate the system through bifurcations and tortuous vasculature and also to deflect the tip within the vessel and thereby direct the pilot wire or other component connected to the tip in a selected direction.

The proximal end 23 of the link is preferably connected to the end of the shield through a rotary connection in the form of a ferrule 25 that is rotatably disposed onto the shield proximally to a ferrule 26. Ferrule 26 is affixed (e.g., bonded) to the distal end of the shield 7 and holds the ferrule 25 longitudinally in place and the proximal end of the link 23 is connected to the ferrule 26 by weldments 28 and 28′. The rotary connection permits different amounts of twisting between the casing and the shield which may be more susceptible to crack under excessive twisting.

The distal end of the helical wire is gated by the distal end 22 of the link 3 that is attached to the last two most distal coils of the helical wire 35 and 36 by weldments 27 and 27′.

The shield 7 has an open distal end and a proximal end that is connected to the second coupling means in the form of the tube 17′ which is connected to a port 51 through a Y connector 52. The Y connector has threaded collars 53 and 54 that, upon tightening, compress seals 55 and 56 reducing their length and causing them to elastically deform and close around the tube 17′ and the pilot wire 9, respectively, or in the absence of a pilot wire, to shut the proximal end of the Y-connector. Similarly, a handle 66 is attached to the tube 17 to facilitate manual rotation and advancement of the casing.

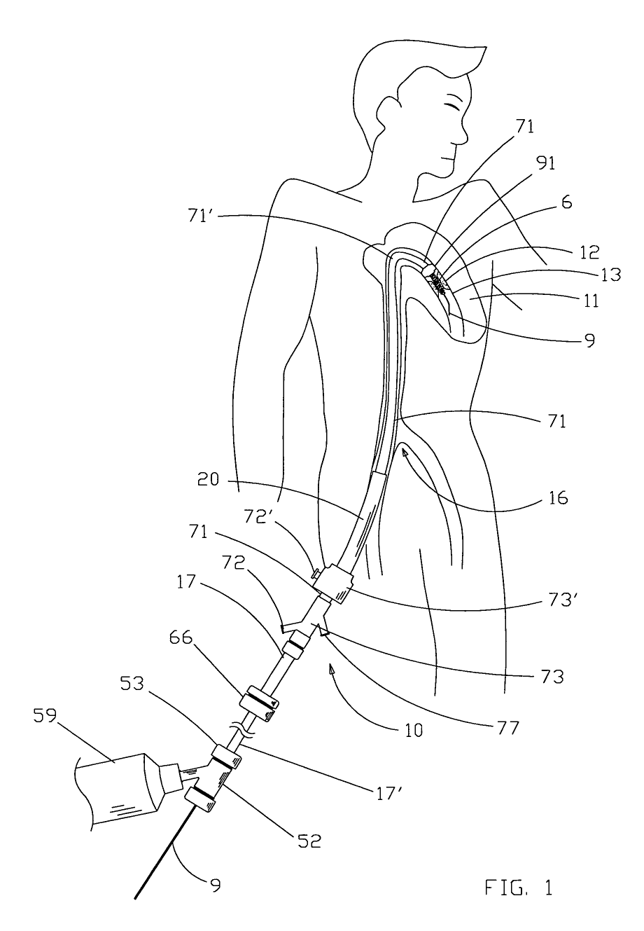

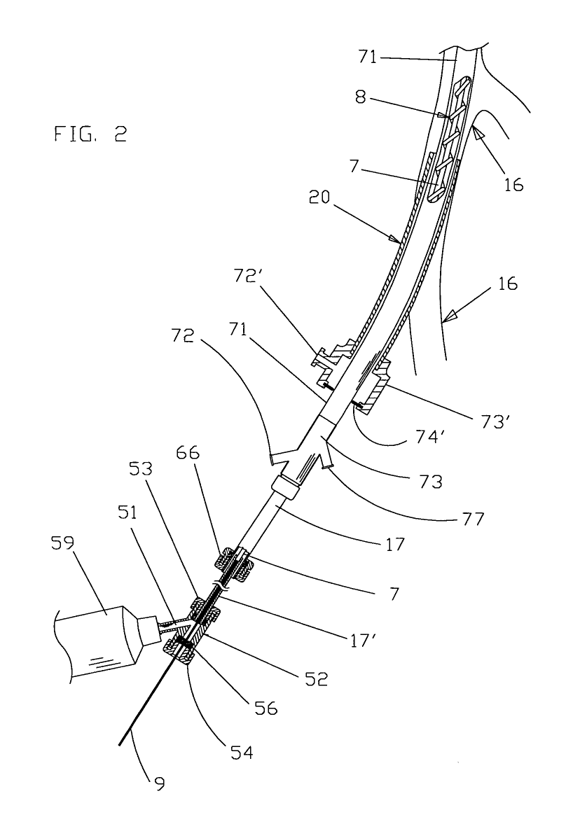

An optional syringe 59 (note FIGS. 1 and 2), preferably with a small diameter piston suitable for generating higher pressures, is connected to the port 51 and can be used to deliver fluid (e.g., saline solution, radio-opaque fluid, drugs) through the distal end of the shield.

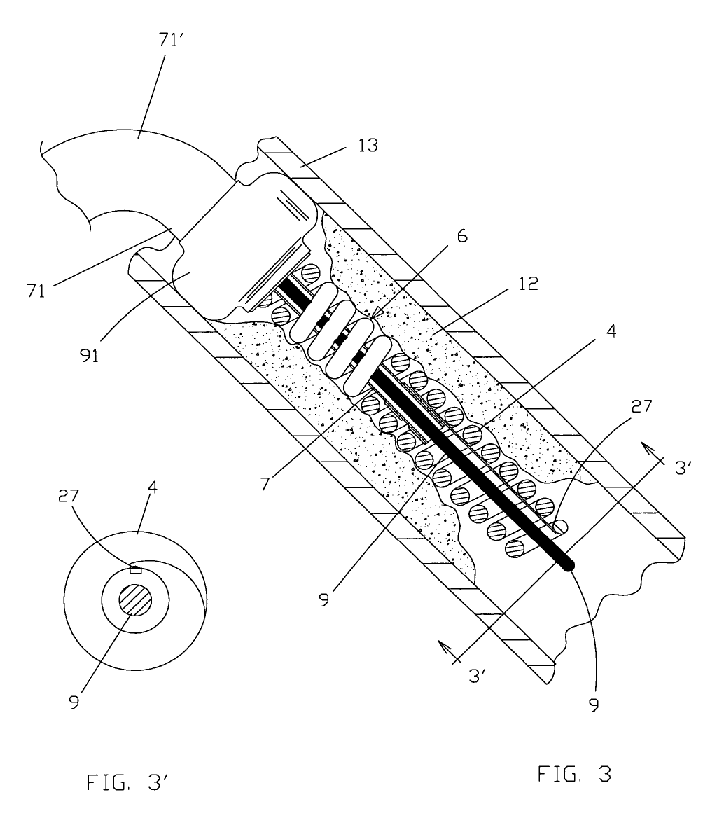

FIGS. 3 and 3′ show side and front views of the distal end of the system, wherein a distal end of the helical wire 4 is ground down to form a smooth inclined plane to ease its penetration and minimize the likelihood of trauma to the vasculature 16 or to the vessel 13.

FIG. 4 shows further details of the casing 8 that is preferably made of a distal section 6 in the form of closely wound coils and a midsection 5 in the form of distantly spaced coils and both are wound from a continuous wire 4 for enhanced integrity. The closely wound coils provide enhanced flexibility whereas the distantly spaced coils provide enhanced torsional and longitudinal rigidity thereby reducing the elastic angular and linear deformation between the distal and proximal ends of the casing under torque and linear loading, respectively. As shown in FIGS. 3 and 4, the wire 4 has a round cross section, however, alternatively, the casing can be wound from a wire with a flattened cross-section an example of which is shown in FIG. 7. Optionally the wire used to form the distal coils can be made of decreasing cross section, or the coils can be ground down, as shown in FIG. 7, to increase the distal tip's flexibility and ability to penetrate and the obstruction.

The tube 17 is connected to the proximal end of the casing preferably by a weldment 49, and serves as an extension of its proximal end with a smooth outside surface that provides a surface suitable for the seal 74 to seal against while the tube 17 is rotated and linearly moved through it. Alternatively, the system can be inserted directly through the introducer 20, in which case the seal 74′ provides the sealing around the tube 17. The casing can be driven (i.e., advanced and rotated), through the tube 17 by the physician.

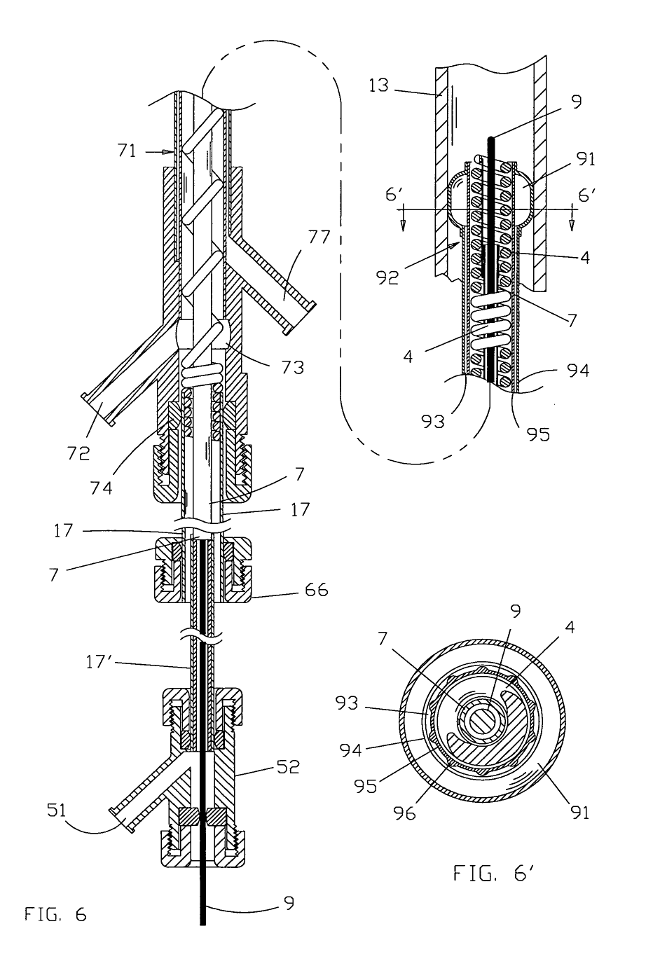

FIGS. 6 and 6′ show side and end views, respectively, of the system disposed in a sleeve 71 with a biasing means in the form of an inflatable distal chamber 91 formed close to the distal end of a sleeve. When inflated, for example by a syringe (not shown), through a port 77 connected to a channel 93 formed between the sleeve's two concentric outer and inner layers 94 and 95, respectively, the chamber bears against the vessel's wall while centering the biasing sleeve in the vessel. Optional longitudinal ridges 96 (that can be extruded as a part of the inner layer) scaffold the channel 93. When deflated, the chamber conforms to the sleeve to minimize interference with its insertion into the vessel. The distal end section of the sleeve can be pre-curved, as shown in FIG. 1 and marked 71′, to direct the distal end of the system into a specific vessel and/or selectively bias it inside the vessel.

An external port 72 that is connectable, for example, to a syringe (not shown) is connected to the flexible sleeve through an annular chamber 73 that is attached to the proximal end of the sleeve. The chamber is equipped with a seal 74 (note FIG. 6) that seals around a smooth outer surface of the tube 17. The sleeve 71 can be inserted into the vasculature directly or through a standard introducer 20 having a port 72′ that is also connectable, for example, to a syringe (not shown), a chamber 73′ and a seal 74′ that seals on the outer surface of sleeve 71 (standard introducers are sold by numerous companies, e.g.: Boston Scientific, Natick, Mass.; Cook, Bloomington, Ind.).

FIGS. 7 and 7′ show the deflectable distal tip of an embodiment of the present invention in which the casing is wound of a flattened wire(the term “flattened-wire”, as used in this application, is derived from a common method of manufacturing such wire by flattening a wire with a round cross section between two adjacent rollers to produce a wire with an elongated cross-section 4′). The distal section of the casing is made from wire 4′ wound on its edge, i.e., its long-axis being approximately perpendicular to a longitudinal axis of the casing. As illustrated in FIGS. 7 and 7′ the most distal coils can be ground down to facilitate starting the threading of the casing into the obstruction and at the same time increasing the flexibility of the distal potion of the distal section 6. The midsection of the casing is made from the wire wound on its side, i.e., its long-axis being approximately parallel to the casing's longitudinal axis.

A system according to the present invention can have different diameters and lengths depending on the size and site of the vessel that it is intended for and on whether the system is to be used percutaneously or intra-operatively. For example, a system that is intended to be introduced percutaneously at the groin area for crossing an obstruction in a coronary vessel preferably utilizes as a pilot wire a standard guidewire with approximately 0.014 inches diameter with a shield having an internal diameter of about 0.020 inches, an outside diameter of 0.26 inches and a length of about 50 inches. The distal portion of the casing can be 10 inches long, the midsection 30 inches long and the tube 17 can be 10 inches long. If the system utilizes a larger diameter pilot wire, such as an 0.035 inches guidewire, the shield and casing dimensions will also increase. If the system is intended for use in peripheral (non-coronary) blood vessels or where direct access to the vessel is gained surgically (intraoperatively), the system can be shorter.

The above mentioned and other modifications and substitutions can be made in the system and in its operation within the spirit of the invention and the scope of the following claims.

1. A flexible guidewire system with a deflectable distal tip comprising:
a flexible tubular shield adapted to slide and rotate over a pilot wire;
a flexible casing disposed over said shield, at least a distal portion of said casing being a helical wire;
a first coupling means connected to said casing;
a second coupling means connected to said shield;
a thin narrow flexible link being disposed in said distal portion of said casing adjacent to one side of an inner surface of said distal portion of said casing, said link having distal and proximal ends that are connected to a distal end of said casing and to a distal end of said shield respectively;
wherein said link pulls and compresses said distal portion of said casing responsive to said first coupling means being displaced relative said second coupling means, causing said helical wire to compress to a larger extent on said one side that is adjacent to said link thereby causing said distal portion of said casing to curve and deflect said distal tip.

2. As in claim 1 wherein a distal end of said shield is connected to said link through a rotary connection.

3. As in claim 2 wherein said proximal end of said shield is hydraulically connected to an external port.

4. As in claim 1, wherein said distal end of said helical wire is gated by said link being attached to at least the two most distal coils of said helical wire.

5. As in claim 1, wherein said helical wire comprises a distal end that is gated by a tube section to which said distal end of said link is connected to.

6. As in claim 1, wherein said casing has a midsection that comprises a helical wire with distantly spaced coils.

7. As in claim 6, wherein said distal portion of said casing and said midsection of said casing are wound of a continuous wire.

8. As in claim 1, wherein said flexible guidewire system with a deflectable distal tip is disposed in a sleeve with a biasing means to deflect said casing in the vessel.

9. As in claim 8, wherein said sleeve comprises a pre-curved distal end section.

10. As in claim 8, wherein said sleeve comprises a selectively inflatable chamber formed at said distal end of said sleeve.
